(19) United States
(12) Patent Application Publication
(10) Pub. No.: US 2010/0059560 A1
(43) Pub. Date: Mar. 11, 2010

(54) PORTABLE CASE FOR STORING AND CARRYING MEDICAL ITEMS

(76) Inventor: Carl Lanum, Crescent City, CA (US)

Correspondence Address: LAW OFFICE OF JERRY D. HAYNES , P.A, P.O. Box 350392, Fort Lauderdale, FL, 33335, US

(21) Appl. No.: 12/208,364

(22) Filed: Sep. 11, 2008

(51) Int. Cl. A61B 19/02; A45F 3/14

(52) U.S. Cl. 224/257; 206/571

(57) ABSTRACT

Disclosed is portable case for storing and carrying medical items. The portable case includes a lower section, an upper section hingedly attached to the lower section, and a locking mechanism. The lower section includes a plurality of compartments and a first set of elastic loops members for holding the medical items. The upper section includes a second set of elastic loops for holding medical items. The locking mechanism is adapted to latch the upper section to the lower section in a closed position of the portable case.

### FIELD OF THE INVENTION

The present invention relates generally to carrying cases, and more particularly, to a portable case for storing and carrying medical items in a convenient manner.

### BACKGROUND OF THE INVENTION

Generally, people suffer from one or more kinds of illness, and accordingly must ingest medicines on a regular basis to fight against the illness. For example, people suffering from medical complications such as diabetes, asthma, heart related complications, and the like are required to constantly take medications to feel better. Besides medicines, various medical devices such as syringes, nebulizers, blood sugar detecting devices, and the like are also required to keep medical complications under check.

Storage of the medicines and the medical devices, hereinafter referred to as “medical items”, has always been cumbersome for people. The medical items, particularly, medicine bottles need to be handled with care as the same are subject to breakage very easily. Further, people on medication need to carry the medical items prescribed to them by a doctor or a physician to almost every place, they intend to go. As such, mobility of such people requiring a constant availability of medication may be severely restricted. Also, the medical items may be hazardous for children, if not stored in a safe custody.

Many attempts have been made to provide a storage means for storage of medical items in order to circumvent the difficulties faced by people in storage and transportation of medical items. Conventional storage means usually comprise a bag or a box in which the medical items are kept. However, such conventional storage means are bulky and difficult to carry. Further, various medical items are available in glass vials or bottles which are subject to breakage when stored inside the conventional storage means. Furthermore, conventional storage means for storage of the medical items lack organizational capabilities to store medical items in a convenient and a user-friendly manner. More specifically, while looking for a particular medical item, a user has to remove all the medical items present in the conventional storage means to locate the particular medical item. This may involve extra time and effort. Moreover, a risk to a human life is also associated in case the particular medical item required during emergency, such as, an attack of asthma, and the like, is not located easily.

Accordingly, there is a need for providing a storage means for medical items, particularly, a portable case capable of storing and carrying the medical items efficiently and in a user-friendly manner. Additionally, there also exists a need for a portable case capable of providing safe custody, to keep the medical items out of the reach of children.

### SUMMARY OF THE INVENTION

In view of the foregoing disadvantages inherent in the prior art, the general purpose of the present invention is to provide a portable case for storing and carrying medical items, configured to include all the advantages of the prior art, and to overcome the drawbacks inherent therein.

Accordingly, an object of the present invention is to provide a portable case for medical items configured to store the medical items in an organized manner in order to provide maximum accessibility to the medical items in less time.

Another object of the present invention is to provide a portable case for storing and carrying medical items with ease and in an efficient manner.

Yet another object of the present invention is to provide a portable case configured to keep the medical items in a safe custody, out of the reach of children.

Yet another object of the present invention is to provide a portable case for transportation of medical items without a fear of breakage of the medical items.

In light of the above objects, in one aspect of the present invention, a portable case for storing and carrying medical items is provided. The portable case comprises a lower section, an upper section hingedly attached to the lower section, and a locking mechanism. The lower section comprises a first base portion, a plurality of first end wall members and a plurality of first sidewall members configuring a first cavity therebetween. Further, the first cavity comprises a plurality of compartments configured therewithin. Further, at least one compartment of the plurality of compartments comprises a first set of elastic loops. The upper section comprises a second base portion, a plurality of second end wall members and a plurality of second sidewall members configuring a second cavity there between. Further, the second base portion comprises a second set of elastic loops. The locking mechanism is configured on at least one second end wall member of the plurality of second end wall members and at least one first end wall member of the plurality of first end wall members. Further, the upper section is adapted to be received on the lower section to assume a closed position and the locking mechanism is adapted to latch the upper section to the lower section in the closed position. The plurality of compartments, the first set of elastic loops and the second set of elastic loops are configured to hold the plurality of medical items.

In another aspect of the present invention, a portable case for storing and carrying medical items is provided. The portable case comprises a handle member configured on an outer side of at least one first end wall member of the plurality of first end wall members. The handle member allows holding of the portable case with ease.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawing, in which:

FIG. 1 illustrates a perspective view of a portable case for storing and carrying medical items, in accordance with an embodiment of the present invention;

FIG. 2 illustrates an exemplary arrangement of plurality of medical items in the portable case of FIG. 1, in accordance with an embodiment of the present invention; and

FIG. 3 illustrates a closed position of the portable case of FIG. 1, in accordance with an embodiment of the present invention.

Like reference numerals refer to like parts throughout the description of several views of the drawings.

### DETAILED DESCRIPTION OF THE INVENTION

The exemplary embodiments described herein detail for illustrative purposes are subject to many variations in structure and design. It should be emphasized, however, that the present invention is not limited to a particular portable case for storing and carrying medical items, as shown and described. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

As used herein, the term “medical items” refer to medicines and medical devices such as medicine bottles, bandages, syringe boxes, ointment tubes, tablets and tablet containers, vials, nebulizers and nebulizer holders, thermometer, blood glucose machines, and the like.

The present invention provides a portable case configured to store a plurality of medical items, hereinafter referred to as ‘medical items’, for easy dispensation therefrom and provide convenient portability. The present invention finds its utility in homes and commercial establishments such as hospitals, clinics, nursing homes and the like. The present invention is beneficial for people from all walks of life who need to carry medical items.

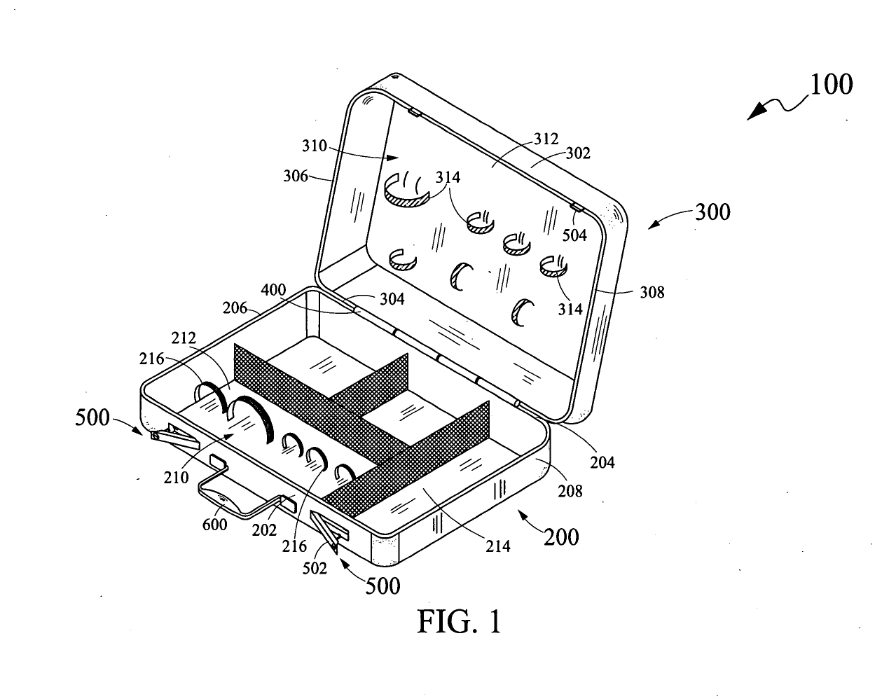

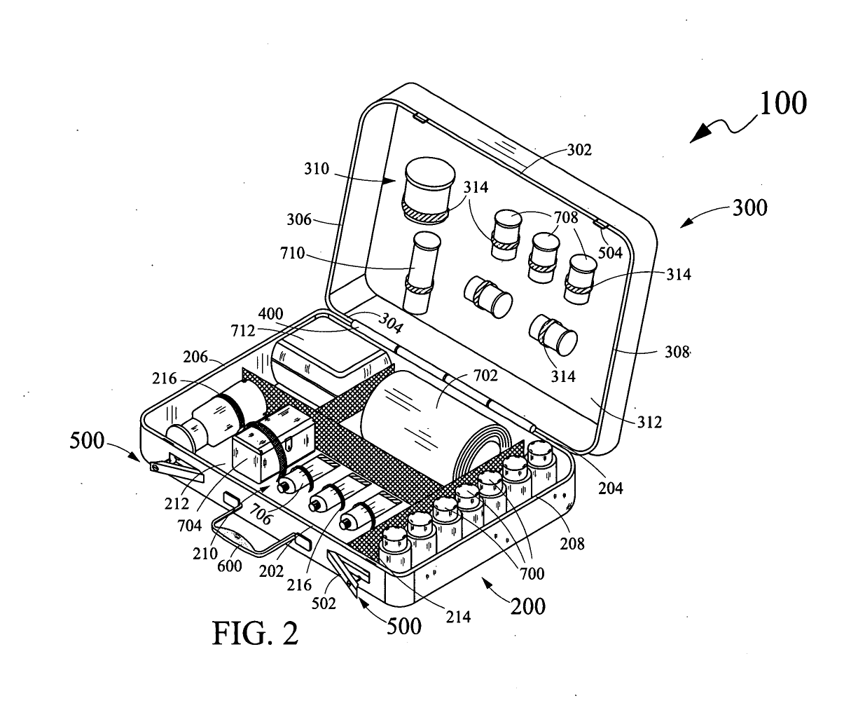

With reference to FIGS. 1 and 2, a portable case 100 for storing and carrying plurality of medical items is illustrated, in accordance with an embodiment of the present invention. More specifically, FIG. 1 illustrates the disclosed portable case 100 and FIG. 2 illustrates an exemplary arrangement of the plurality of medical items in the portable case 100. The portable case 100 includes a lower section 200 and an upper section 300 hingedly attached to the lower section 200.

As illustrated in FIGS. 1 and 2, the lower section 200 and the upper section 300 may be dimensionally similar. The lower section 200 includes a first base portion (not shown); a plurality of first end wall members, such as first end wall members 202, 204; and a plurality of first sidewall members, such as first sidewall members 206, 208. The first base portion, the plurality of first end wall members, and the plurality of first sidewall members collectively configure a first cavity 210 therebetween. The first cavity 210 may be utilized for storing the plurality of medical items.

The lower section 200 further includes a first lining 212 attached to an inner surface of the first cavity 210. More specifically, the inner surface of the first cavity 210 may be covered by the first lining 212. In an embodiment of the present invention, the first lining 212 may be composed of a suitable material such as rubber foam. However, it will be evident to a person skilled in the art that other types of lining may be utilized in place of rubber foam, such as a soft fibrous material, a thermoplastic material, and the like.

The first cavity 210 of the lower section 200 may comprise a plurality of compartments, such as a compartment 214. The plurality of compartments is capable of holding various medical items such as medicine bottles, medical apparatuses, and the like, therein. Further, the lower section 200 includes a first set of elastic loops, such as an elastic loop 216 for holding the medical items. The positioning of medical items within the plurality of compartments and the first set of elastic loops is explained later in conjunction with FIG. 2. The first set of elastic loops, such as the elastic loop 216 may be made of elastic material and may have a loop-like structural configuration for holding the medical items in an upright position. The medical items may be held in place by means of the first set of elastic loops, such as the elastic loop 216 to avoid any dislodgment of the medical items during the movement of the portable case 100. In an embodiment of the present invention, the first set of elastic loops, such as the elastic loop 216 are secured upon the first lining 212. In another embodiment of the present invention, the first set of elastic loops, such as the elastic loop 216 are secured with the first base portion of the lower section 200 and slots may be made in the first lining 212 to allow the first set of elastic loops, such as the elastic loop 216 to pass through the first lining 212.

Further, the portable case 100 includes the upper section 300 hingedly attached to the lower section 200. The upper section 300 includes a second base portion (not shown); a plurality of second end wall members, such as second end wall members 302, 304; and a plurality of second sidewall members, such as second sidewall members 306, 308. The second base portion, the plurality of second end wall members and the plurality of second sidewall members collectively configure a second cavity 310. The second cavity 310 may be utilized for storing the medical items from the plurality of medical items. The second cavity 310 of the upper section 300 includes a second lining 312 extending throughout the second cavity 310. The material of construction of the second lining 312 may be similar to that of the first lining 212. The first lining 212 and the second lining 312 provides a padded interior to the lower section 200 and the upper section 300 respectively, in order to prevent breakage of the medical items.

Further, the upper section 300 includes a second set of elastic loops, such as an elastic loop 314 for holding the medical items. The second set of elastic loops may be similar to the first set of elastic loops. Each elastic loop of the second set of elastic loops, such as the elastic loop 314 may be made of an elastic material, in order to hold the medical items inserted therewithin in a secure and an upright position. Moreover, each elastic loop of the second set of plurality of elastic loops may be of different sizes to enclose the medical items of varied dimensions.

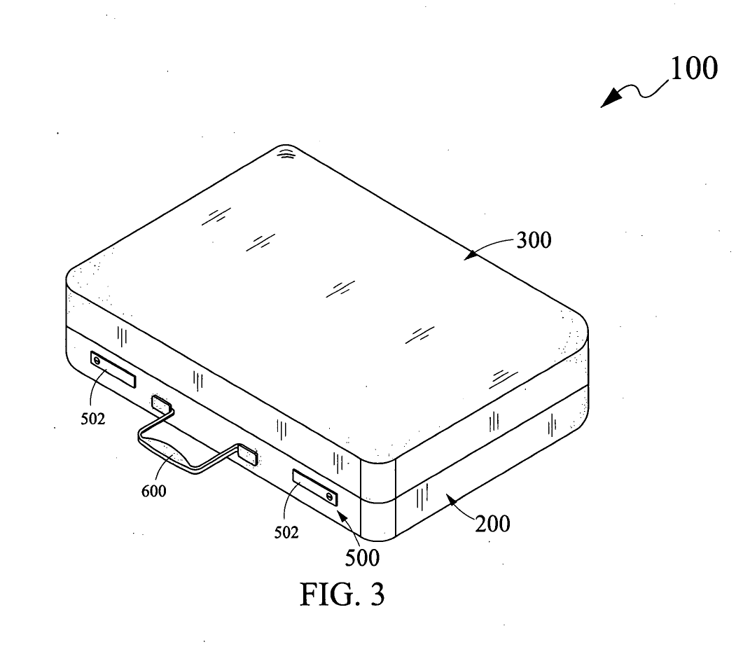

The upper section 300 is hingedly attached to the lower section 200. More specifically, a peripheral edge of the first end wall member 204 of the lower section 200 and a peripheral edge of the second end wall member 304 of the upper section 300 are pivotally attached to each other by a hinge member 400. Pivotal engagement of the lower section 200 and the upper section 300 by means of the hinge member 400 allows the portable case 100 to assume a plurality of positions between a completely open position and a completely closed position. The open position of the portable case 100 is depicted in FIGS. 1 & 2. When in the open position, the portable case 100 enables a user to access the medical items therefrom. The closed position of the portable case 100 may be explained in detail in conjunction with FIG. 3.

Referring to FIG. 2, the portable case 100 further includes at least one locking mechanism 500 configured on the lower section 200 and the upper section 300. More specifically, the at least one locking mechanism 500 includes a latching section 502 configured on the lower section 200 of the portable case 100 and a tongue portion 504 configured on the upper member 300, of the portable case 100. The tongue portion 504 may be mounted on the second end wall member 302 of the upper section 300 and is adapted to releasably engage with the latching member 502 disposed on the first end wall member 202 of the lower section 200. FIGS. 1 & 2 illustrate two locking mechanisms such as the locking mechanism 500 configured on the portable case 100. It will be apparent to those skilled in the art that other locking mechanism for releasably engaging the lower section 200 and the upper section 300 may be employed, and the locking mechanism 500 as set forth herein are for the purpose of illustration and are not to be construed as limiting. In an embodiment of the present invention, the locking mechanism 500 includes a notch flap (not shown) extending from the upper section 300 readily adapted to engage notch catch (not shown) mounted on the lower section 200. In another embodiment of the present invention, the locking mechanism 500 may be a snap fit mechanism.

Further, the portable case 100 includes a handle member 600 for convenient carriage and handling of the portable case 100. The handle member 600 is mounted on an outer side of the first end wall member 202 of the lower section 200. In an embodiment of the present invention, the portable case 100 may also include a shoulder strap (not shown) configured on the outer side of the first end wall member 202 to carry the portable case 100.

As illustrated in FIG. 2, the lower section 200 and the upper section 300 is capable of holding the plurality of medical items. The plurality of medical items such as a plurality of medicine bottles 700, a bandage 702, a syringe box 704, a plurality of ointment tubes 706, a plurality of tablet containers 708, a vial 710, and a nebulizer holder 712 may be held in the plurality of compartments, such as the compartment 214, the first set of elastic loops such as the elastic loop 216 and the second set of elastic loops such as the loop 314. It will be apparent to those skilled in the art that the plurality of medical items as illustrated in FIG. 2 and the arrangement of the medical items in the portable case 100 as set forth herein are for the purpose of illustration and are not to be construed as limiting. The portable case 100 is adapted to hold and carry different types of medical items.

FIG. 3 illustrates the closed position of the portable case 100, in accordance with an embodiment of the present invention. The portable case 100 assumes the closed position when the upper section 300 comes in contact with the lower section 200 from all sides, to enclose the medical items stored within the portable case 100. The locking mechanism 500 is capable of securing the portable case 100 in the closed position.

In use, the medical items are removably secured within the portable case 100. More specifically, the medical items are placed in appropriate compartments from the plurality of compartments, such as the compartment 214, appropriate holding members from the first set of elastic loops, such as the elastic loop 216, and appropriate elastic loops from the second set of elastic loops, such as the elastic loop 314. After the medical items have been placed within the portable case 100, the upper section 300 may be received over the lower section 200, to allow the portable case 100 to assume the closed position. Thereafter, the locking mechanism 500 is operated to securely attach the upper section 300 and the lower section 200. More specifically, the tongue portion 504 readily engages with the latching member 502 to make the portable case 100 secure. The portable case 100 may be moved from one place to other with the help of the handle member 600 disposed on the lower section 200 of the portable case 100. Moreover, when a particular medical item from the plurality of medical items is required to be accessed, the locking mechanism 500 may be operated in a manner such that the tongue portion 504 is released from the latching member 502 and the portable case 100 assumes the open position. Accordingly, the particular medical item may be accessed and removed from the portable case 100.

The portable case 100 may have different sizes and shapes. In an embodiment of the present invention, the portable case 100 may be about 16 inches in length, about 12 inches in width and about 8 inches in height. Further, the portable case 100 may be about 3 pounds in weight. The present invention provides a sleek and a lightweight storage means for storing and carrying the medical items.

The portable case of the present invention serves to hold and carry the medical items in an effective manner without causing breakage of the medical items. Further, the portable case provides a mean to store the medical items in a safe custody out of the reach of the children.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and thereby enable others skilled in the art to best utilize the present invention and various embodiments with various modifications as are suited to the particular use contemplated. It is understood that various omissions and substitutions of equivalents are contemplated as circumstances may suggest or render expedient, but such are intended to cover the application or implementation without departing from the spirit or scope of the claims of the present invention.

What is claimed is:

1. A portable case for storing and carrying a plurality of medical items, the portable case comprising:
a lower section having a first base portion, a plurality of first end wall members and a plurality of first sidewall members configuring a first cavity therebetween, the first cavity comprising a plurality of compartments, at least one compartment of the plurality of compartments comprising a first set of elastic loops attached to the first base portion;
an upper section hingedly attached to the lower section, the upper section having a second base portion, a plurality of second end wall members and a plurality of second sidewall members configuring a second cavity therebetween, the second cavity comprising a second set of elastic loops attached to the second base portion; and
a locking mechanism configured on at least one second end wall member of the plurality of second end wall members and at least one first end wall member of the plurality of first end wall members,
wherein the upper section is adapted to be received on the lower section to assume a closed position, and
wherein the locking mechanism is adapted to latch the upper section to the lower section in the closed position, and
wherein the plurality of compartments, the first set of elastic loops and the second set of elastic loops are configured to hold the plurality of medical items.

2. The portable case of claim 1, wherein the lower section comprises a first lining configured within the first cavity of the lower section.

3. The portable case of claim 1, wherein the upper section comprises a second lining configured within the second cavity of the upper section.

4. The portable case of claim 1, wherein the lower section further comprises a handle member configured on an outer side of the at least one first end wall member of the plurality of first end wall members.

5. The portable case of claim 1, wherein the locking mechanism comprises
a tongue portion configured on the upper section, and
a latching member configured on the lower section, the latching member adapted to releasably engage the tongue portion for locking the portable case in the closed position.

6. The portable case of claim 1, further comprising a shoulder strap configured on an outer side of the at least one first end wall member of the plurality of first end wall members.
